(19) United States
(12) Patent Application Publication
(10) Pub. No.: US 2016/0103144 A1
(43) Pub. Date: Apr. 14, 2016

(54) BLOOD BASED METHODS OF ASSESSING ADOLESCENT DEPRESSION IN A SUBJECT

(71) Applicant: True Health Diagnostics, LLC, Frisco, TX (US)

(72) Inventors: William S. Harris, Sioux Falls, SD (US); James V. Pottala, Sioux Falls, SD (US); Andrew B. Christianson, Sioux Falls, SD (US)

(21) Appl. No.: 14/974,239

(22) Filed: Dec. 18, 2015

Related U.S. Application Data
(63) Continuation of application No. 12/274,764, filed on Nov. 20, 2008, now abandoned.

(51) Int. Cl. G01N 33/92; G06F 19/00

(52) U.S. Cl. CPC G01N 33/92; G06F 19/3431; G01N 2800/304; G01N 2800/38; G01N 2405/00; G01N 2570/00

(57) ABSTRACT

The present invention provides blood based methods for assessing adolescent depression in a subject.

### CROSS REFERENCE

This application is a continuation application of U.S. patent application Ser. No. 12/274,764 filed Nov. 20, 2008, the entirety of which is incorporated herein by reference and relied upon.

### BACKGROUND

Currently there are no reliable blood-based diagnostic methods for identifying adolescents suffering from depression. The rising incidence of adolescent depression and the severe consequences to both individuals and society make clear the need for such diagnostic methods.

### SUMMARY OF THE INVENTION

The present invention provides methods for assessing adolescent depression in a subject, comprising:

(a) measuring an amount of one or more fatty acids in tissue from an adolescent subject;

(b) comparing the amount of the one or more fatty acids to a control; and

(c) determining a probability that the adolescent subject has depression based on the comparison.

In a second aspect, the present invention provides methods for determining the fatty acid composition of a tissue membrane, comprising:

(a) subjecting a tissue sample to at least one freeze-thaw cycle;

(b) methylating fatty acids in the tissue sample to produce fatty acid methyl esters (FAMEs) by treatment with a boron-trifluoride methanol solution;

(c) extracting FAMEs from the tissue sample using a hexane solvent;

(d) separating the FAMEs by gas chromatography in a fused silica capillary column;

(e) determining retention times of the FAMEs in the column by means of a flame ionization detector (FID); and

(f) comparing the retention time to a FAME standard; and

(g) calculating FAME response factors for each FAME in the sample based on a FAME standard.

Various embodiments of the different aspects of the invention are described in detail below.

### DETAILED DESCRIPTION OF THE INVENTION

In one aspect, the present invention provides methods for assessing adolescent depression in a subject, comprising

(a) measuring an amount of one or more fatty acids in a tissue sample from a subject at risk of suffering from adolescent depression;

(b) comparing the amount of the one or more fatty acids to a control; and

(c) determining a probability that the adolescent subject has depression based on the comparison.

The inventors have discovered that a subset of tissue fatty acids can be used in the diagnosis of adolescent depression. The methods of the invention can be used, for example, to assess the probability that an adolescent suffers from depression. The methods can thus assist in the diagnosis of depression by adding an objective measure to the usual subjective clinical signs used to diagnose depression. More confident diagnosis can lead to earlier and more aggressive treatment that can reduce the burden of adolescent depression both for patients and for society.

As used herein, depression is a disorder characterized by a pervasive low mood and loss of interest or pleasure in usual activities. Symptoms of depression can vary; they include, but are not limited to pervasive low mood, loss of interest in activities previously of interest, feelings of worthlessness, inappropriate guilt or regret, helplessness or hopelessness, poor concentration and memory, withdrawal from social situations and activities, thoughts of death or suicide, insomnia, appetite decrease, weight loss, fatigue, headaches, digestive problems, chronic pain, agitation, sluggishness, delusions, irritability, loss of interest in school, decline in academic performance, clinginess, attention demanding behavior, dependency, insecurity, drug abuse, alcohol abuse, and disruptive behavior.

As used herein, an “adolescent” is one that is between the period of childhood and adulthood; in one embodiment, an adolescent is a subject in which puberty onset has begun, but the subject has not yet reached the age of 21. In another embodiment, the adolescent is between the ages of 10 and 21; in another embodiment, between 13 and 19 years of age.

The methods comprise determining an amount of the one or more fatty acids in a tissue sample obtained from an adolescent, or an adolescent subject at risk of suffering from depression, such as an adolescent showing one or more symptoms of depression. In various embodiments, the tissue sample can comprise or consist of plasma, serum, red blood cells, platelets, white blood cells or whole blood. Methods for preparation of fatty acids from tissue samples are known in the art (see, for example, Block et al., Atherosclerosis 2008 April; 197(2):821-8), and exemplary such methods are disclosed below.

Determining an amount of the fatty acids can comprise any suitable measurement, including but not limited to determining an amount of a fatty acid for the tissue type as a weight percent of total fatty acids, a molar percentage of total fatty acids, a concentration in the tissue type, etc.

In another embodiment, the one or more fatty acids are selected from the group consisting of myristic acid, palmitic acid, palmitelaidic acid, palmitoleic acid, stearic acid, elaidic acid, oleic acid, linolelaidic acid, linoleic acid, gamma linolenic acid, cis-11-eicosenoic acid, alpha-linolenic acid, cis-11,14-eicosadienoic acid, cis-8-11-14-eicosatrienoic acid, arachidonic acid, lignoceric acid, eicosapentaenoic acid (EPA), nervonic acid, docosatetraenoic acid, n-6 docosapentaenoic acid, n-3 docosapentaenoic acid, and docosahexaenoic acid (DHA). In various further embodiments, the one or more fatty acids comprise 2, 3, 4, 5, 6, 7, 8, 9, 10, 11, 12, 13, 14, 15, 16, 17, 18, 19, 20, 21 or all 22 of the recited fatty acids.

In a further embodiment, the one or more fatty acids are selected from the group consisting of linolelaidic acid, palmitelaidic acid linoleic acid, arachidonic acid, alpha-linolenic acid, myristic acid, gamma-linolenic acid, docosatetraenoic acid, palmitoleic acid, and cis-8-11-14-eicosatrienoic acid. In various further embodiments, the one or more fatty acids comprise 2, 3, 4, 5, 6, 7, 8, 9, 10, 11 or 12 of the recited fatty acids. In another embodiment, the one or more fatty acids comprise linolelaidic acid, linoleic acid, arachidonic acid, palmitoleic, cis-8-11-14-eicosatrienoic acid, alpha-linolenic acid, myristic acid, and/or docosatetraenoic acid; in one variation of this embodiment, the subject is an African-American adolescent and the one or more fatty acids comprise 1, 2, 3, 4, 5, 6, 7, or 8 of the recited fatty acids.

In another embodiment, the adolescent is Caucasian, and the one or more fatty acids comprise linolelaidic acid, palmitelaidic acid, linoleic acid, arachidonic acid, palmitoleic, cis-8-11-14-eicosatrienoic acid, alpha-linolenic acid, myristic acid, gamma-linolenic acid, and docosatetraenoic acid.

In various other embodiments, the methods comprise determining one or more of the fatty acid combinations disclosed in Table 4. In another embodiment, the methods comprise determining an amount of all fatty acids in the tissue sample.

The methods further comprise comparing the amount of the one or more fatty acids to a control. As used herein, a “control” is any means for normalizing the amount of the one or more fatty acids (FA) being measured from the subject to that of a standard. In one embodiment, the control comprises a pre-defined fatty acid level or levels from a normal individual or population, or from an individual or population of subjects suffering adolescent depression. In another embodiment, the control comprises a known amount of the one or more FA from the tissue type being sampled in adolescent depression or non-adolescent depression subjects. In another embodiment, the amount of a single FA, such as palmitelaidic acid, DHA, alpha-linolenic acid, gamma-linolenic acid, palmitoleic acid, oleic acid, or cis-8-11-14-eicosatrienoic acid, is compared to the amount of the single fatty acid in a control sample, wherein the difference between the single fatty acid content of the two samples can be used to determine a probability of adolescent depression in the subject. In other embodiments, the amounts of two or more FA (such as any combination of the above) are directly compared to a control to determine the probability of adolescent depression in the subject.

In another embodiment, the comparison may comprise adjusting the amount of the one or more fatty acids by an appropriate weighting coefficient, hereinafter referred to as “a beta coefficient”. In one embodiment, the method comprises (a) multiplying the amount of the individual one or more fatty acids (expressed as a percent of total fatty acids in the sample) by a predetermined beta coefficient to produce an individual fatty acid score; and (b) summing the individual fatty acid scores to produce a risk score. This risk score can then be used in another equation to determine the probability that a given subject has adolescent depression or is at higher risk for developing adolescent depression than a person with a lower score. As will be understood by those of skill in the art based on the teachings herein, beta coefficients can be determined by a variety of techniques and can vary widely. In one example of determining appropriate beta coefficients, multivariable logistic regression (MLR) is performed using the fatty acids values found within two groups of patients, for example, one with and one without adolescent depression. There are several methods for variable (fatty acids) selection that can be used with MLR, whereby the fatty acids not selected are eliminated from the model and the beta coefficients for each predictive fatty acid remaining in the model are determined. These beta coefficients are then multiplied by the fatty acid content of the sample (expressed as a percent of total fatty acids in the sample) and then summed to calculate a weighted score. The resulting score (“the risk score”) can then be compared with a particular cutoff score (ie: a threshold), above which a subject is diagnosed as suffering from adolescent depression or not suffering from adolescent depression.

In various further embodiments, the fatty acid data (including, but not limited to, percent total of the individual one or more fatty acids, molar percentage of total fatty acids in the tissue, a concentration in the tissue, etc.) are subjected to one or more alternative transformative analyses, including but not limited to generalized models (e.g. logistic regression, generalized additive models), multivariate analysis (e.g. discriminant analysis, principal components analysis, factor analysis), and time-to-event “survival” analysis to produce a modified score; wherein the modified score can be used to determine a probability for adolescent depression.

In exemplary embodiments of the methods, Multivariable Logistic Regression (MLR) or Discriminant Analysis (DA) can be used as classification methods for determining the probability of adolescent depression based on FA amounts. Fatty acids are assumed to be independent in MLR and to be inter-correlated in DA. The fatty acids are the predictor variables (Xᵢ's) and the probability for adolescent depression is the outcome (Y). The probability is a continuous variable which can be dichotomized into two levels such as a binomial (0=No Disease, 1=Disease) response or several discrete levels in an ordinal (0, 1, 2, etc. for different levels of severity of adolescent depression) response. The fatty acids could have linear or nonlinear relationships with the disease outcome.

In MLR a single continuous variable—a riskscore—can be calculated (equation 1) for each subject as the linear combination of the risk factor coefficients (βᵢ's) multiplied by the subject's fatty acids abundances (xᵢ's) expressed as a percent of total fatty acids in the sample.

riskscore=β₀+β₁x₁+β₂x₂ . . . +βₚxₚ Equation 1:

Then the riskscore can be used in the logit function (equation 2) to determine the probability of adolescent depression.

$$prob . of adolescent depression = \frac{1}{1 + {e}^{- ( riskscore )}} \qquad Equation 2$$

The probability of adolescent depression is then dichotomized above a threshold value (e.g. >0.5) for a positive test, in which case the subject would be placed in the higher risk category. Alternatively, subjects can be discretized into test outcome groups (e.g. low, medium, or high risk, or to quartiles of risk, etc.)

DA can be used in a similar manner as MLR, however, the analysis is more complex due to the fatty acids being inter-correlated. The goal in DA is to minimize the distance between the observation and a group centroid in multidimensional space; then the generalized squared distance—a risk score—can be converted into a probability of membership for each outcome level. DA can be used with linear or quadratic decision boundaries.

In principal components analysis, all or a subset of the fatty acids may be used and no assumption regarding independence of variables is made. PCA results can be derived either after mean-centering or standardizing the data. The resulting scores are independent by definition and may be qualified for inclusion into generalized models (e.g. multivariable logistic or ordinal logistic regression) and their importance is based upon their contribution to overall variance. In this embodiment an eigenvector describes the relative contribution of each dimension to overall variability. Individual fatty acid loading values in each dimension (1 through x, where x is the total number of fatty acids analyzed) are used to derive a PCA-Score for each dimension summing the product of each fatty acid percent composition by each FAs loading value for that dimension. The summed value is the PCA-Score(n) where n is the dimension number. The PCA-Score(n) itself can be used for risk prediction. A subset or all of the dimensions can be used to classify an individual in multidimensional space. For example when 3 principal components are used it results in a 3-dimensional object. There can be certain locations within the object where the probability of adolescent depression is above a threshold and these locations are positive test outcomes.

Alternatively the score from all or a subset of the dimensions may be entered into generalized models (e.g. multivariable logistic or ordinal logistic regression) for risk prediction. In evaluating an individual for categorization, the subject's FA is mean centered or standardized using some control values then PCA-scores are derived using some control loading values. Finally the resulting PCA-Score(n) are multiplied by control beta coefficients and summed to create a ‘risk score’. This risk score is entered into the logit equation, to determine the probability of having adolescent depression.

In another embodiment that can be combined with any of the other embodiments herein, the resulting measurements/risk scores/modified risk scores are adjusted for confounding factors such as age, race, gender, socioeconomic status, blood pressure, diabetic status, lipid profile, and/or body-mass index (BMI).

One skilled in the art will understand, based on the teachings herein, that given a set of measurements, such as the FA levels for a given tissue sample from a subject, and given these measurements obtained from a particular set of patients (such as those with adolescent depression) and from a group of ‘normal’ subjects, then there are many techniques for deriving a means for classifying an individual with adolescent depression. Where the method comprises measurement of the amount of two or more FA, the specific differences in the levels of individual FA compared to control matter in the analysis, but it is the overall pattern that is important in distinguishing subjects with and without depression; any individual fatty acid that is a part of the pattern may be higher or lower than the control for one individual and possibly the reverse in another individual, and yet the overall pattern could still distinguish cases from controls. In some embodiments, what matters are whether the sum of the weighted values (the risk score) is greater than or less than a defined threshold. It can be the risk score or the specific level of each specific FA in the predictive classification means that determines how the patient is classified

The methods of the invention further comprise determining a probability that the adolescent subject has depression based on the comparison in step (b) of the method. The “probability” referred to means an increased probability for adolescent depression relative to control, and preferably a statistically significant increased probability for adolescent depression relative to control. The inventors have discovered that a subset of tissue fatty acids can be used in the diagnosis of adolescent depression. The methods of the invention can be used, for example, to assess the probability that an adolescent suffers from depression. The methods can thus assist in the diagnosis of depression by adding an objective measure to the usual subjective clinical signs used to diagnose depression. More confident diagnosis can lead to earlier and more aggressive treatment that can reduce the burden of adolescent depression both for patients and for society.

In a second aspect, the present invention provides methods for determining the fatty acid composition of a tissue membrane, comprising

(a) methylating fatty acids in the tissue sample to produce fatty acid methyl esters (FAMEs) by treatment with a boron-trifluoride methanol solution;

(b) extracting FAMEs from the tissue sample using a hexane solvent;

(c) separating the FAMEs by gas chromatography in a fused silica capillary column;

(d) determining retention times of the FAMEs in the column by means of a flame ionization detector (FID); and

(e) comparing the retention time to a FAME standard; and

(f) calculating FAME response factors for each FAME in the sample based on a FAME standard.

The methods of this second aspect of the invention can be used, for example, to carry out the methods of the invention, as well as to analyze the fatty acid composition of any tissue type of interest.

Any suitable tissue sample can be used for fatty acid analysis, including but not limited to those described above for use in determining a probability of adolescent depression. In one embodiment, the methods may comprise subjecting the tissue sample to one or more freeze-thaw cycles prior to step (a). Any suitable number of freeze-thaw cycles can be used; it is within the level of skill in the art to determine specific freeze-thaw conditions for a given analysis. Similarly, it is within the level of skill in the art, based on the teachings herein, to determine suitable methylation conditions, gas chromatography conditions, and FID conditions for a given assay. Exemplary conditions are provided below.

In one embodiment, calculating FAME response factors for each FAME comprises:
(i) identifying the area counts for a reference fatty acid in the FAME standard and dividing the area counts by the known area percent of the reference fatty acid in the FAME standard; this establishes a certain number of area counts per percent composition in the FAME standard (hereafter the reference ratio)
(ii) multiplying the known percent composition of all other fatty acids in the FAME standard by the reference ratio to generate adjusted area counts
(iii) dividing the observed area counts for each fatty acid in the FAME standard by the adjusted area counts to generate a response factor.
(iv) multiplying the observed area counts for each fatty acid in a sample by its own response factor to produce adjusted area counts for each fatty acid in the sample
(v) summing all of the adjusted area counts for all of the fatty acids in the sample and then dividing the adjusted area counts for each individual fatty acid by the total adjusted area counts in order to express each fatty acid in the sample as a percent of total fatty acids in the sample.

In one example of this embodiment, assume that the area counts for a reference FA in the FAME standard (such as C16:0, palmitic acid) are found to be 1000, and the known percent composition of palmitic acid in the FAME standard is 10% of total fatty acids. The area counts are divided by the percent composition (1000/10) to give a value of 100 area counts per 1 percent of fatty acids. This value is then applied to all other fatty acids in the FAME standard. For example, if the known percent composition of oleic acid is 20% in the FAME standard then the expected area counts for oleic acid should be 20×100, or 2000 area counts. However, say the observed area counts for oleic acid are 1900 counts. This is artificially low and needs to be adjusted upwards. Accordingly, the observed area counts are divided by the expected area counts (1900/2000=0.95) to generate a response factor for oleic acid, here 0.95. This is done for all fatty acids in the FAME standard. The response factors thus determined are then applied to the observed fatty acid area counts in the unknown samples. For example, assume that a hypothetical sample contained only 3 fatty acids (impossible in vivo, but illustrative). Assume that the area counts of oleic acid, linoleic acid and stearic acid in the unknown were 100, 200 and 400 counts, respectively, for a total of 700 area counts. Without adjustment for response factors, the percent composition would be 14%, 28% and 56%. Assume however, that the response factors (as determined from running the FAME standard in the same batch) were 0.90, 1.05 and 0.98, respectively. Adjusting the observed area counts by the response factors would give 90, 210, and 392 adjusted area counts. Summing these three=692 total adjusted area counts. The percent compositions based on the adjusted area counts then become 13%, 30% and 57%.

### Examples

### Methods

### 1) Exemplary Sample Collection

1) Typically, tissue samples are collected for analysis or storage in the following way:
   i) A sample of tissue of interest (typically blood) is collected by venipuncture Whole blood or fractions thereof can be drawn into any anti-coagulant blood collection tube such as sodium citrate, EDTA, or heparin.
   ii) An aliquot is taken from the red blood cell (RBC) pellet after centrifugation to separate plasma from RBCs.
   iii) The aliquot is placed into a micro centrifuge tube, or other suitable storage tube and stored at −80° C.
   iv) Tissue samples are sent on dry ice via an overnight shipment to the processing laboratory (if needed).

### 2) Exemplary Sample Preparation

1) Tissue samples (RBCs in this example) are prepared for GC analysis in the following way:
   i) Thaw the sample completely.
   ii) Bring BF₃-methanol (14%, v/v) solution to RT prior to analysis.
2) Preparation of Instruments
   i) Allow the gas chromatography apparatus and detection apparatus to warm up at least 30 minutes prior to an injection, fill hexane rinse vials for the syringe on the auto injector.
   ii) Verify the proper operation of the gas chromatograph by injection of suitable standard. The appearance of peaks at the expected retention times is indicative of proper operation of the instrument.
3) Work up Procedure
   i) Methylation of red blood cells (RBCs)
      (1) Transfer 25 μL of RBCs to a 2 mL vial.
      (2) Add 250 μL of BF3-methanol, 14% along with 250 μL of EMD hexane and tightly cap the vial.
      (3) Heat the vial for 10 minutes at 100° C.
   ii) FAME extraction
      (1) Add 250 μL of HPLC water, shake the vial for 30 seconds, and centrifuge at 3000 RPM for 3 minutes to separate layers.
      (2) Transfer 50 μL of the hexane supernatant to a GC injection vial and use thumb pressure to apply a snap cap.
      (3) Samples are analyzed by GC-FID on the day they are prepared or kept at −80° C. until analyzed.

### Exemplary Analytical Reference Materials (for RBC FA Preps)

GLC FAME Standard
(a) Various gravimetric percentages of known FAMEs prepared by Matreya (Pleasant Gap, Pa.) and supplied in sealed vials of 10 mg/mL in decane. The stock solution is diluted with 9 mL of hexane to an approximate concentration of 1 mg/mL. A working solution is created by again diluting the stock solution 1:10 with hexane to an approximate concentration of 0.1 mg/mL. The stock and working solutions are stored at −20° C. The exact composition of the GLC FAME standard is as follows:

TABLE 1

| | GLC FAME | |
|---|---|---|
| | External | |
| FAME # | Standard | Weight % |
| 1 | C14:0 | 1 |
| 2 | C16:0 | 16 |
| 3 | C16:1n7t | 1 |
| 4 | C16:1n7 | 2 |
| 5 | C18:0 | 14 |
| 6 | C18:1t | 1 |
| 7 | C18:1n9 | 10 |
| 8 | C18:2n6tt | 1 |
| 9 | C18:2n6 | 12 |
| 10 | C20:0 | 2 |
| 11 | C18:3n6 | 1 |
| 12 | C20:1n9 | 2 |
| 13 | C18:3n3 | 1 |
| 14 | C20:2n6 | 2 |
| 15 | C22:0 | 1 |
| 16 | C20:3n6 | 3 |
| 17 | C20:4n6 | 12 |
| 18 | C24:0 | 1 |
| 19 | C20:5n3 | 3 |
| 20 | C24:1n9 | 1 |
| 21 | C22:4 | 3 |
| 22 | C22:5n6 | 1 |
| 23 | C22:5n3 | 3 |
| 24 | C22:6n3 | 6 |
| | | 100 |

Frozen (−80° C.) high and low omega-3 content pools of RBCs exist as controls, and are worked up with each batch.
Low Control: Pooled red blood cells supplied by a non supplemented omega-3 individual. Target value is an OM31 lower than 4%.
High Control: Pooled red blood cells supplied by a supplemented omega-3 individual. Target value is an OM31 higher than 8%.

Analysis of FAMEs by GC-FID
iii) FAME samples that have been worked up and are contained in labeled GC vials are loaded onto the GC auto sampler.
iv) A batch file is generated in the GCsolution software based on the samples to be run. A typical batch consists of standards, controls, and unknown samples (20-30).
v) Sufficient supplies (2 mL vials, pipette tips, GC vials, etc.) are prepared for the analytical run.
vi) Each vial is labeled with its corresponding sample name and ID. The sequenced order of the batch run is as follows:
   (1) GLC FAME standard
   (2) Low Control
   (3) High Control
   (4) Unknown samples
vii) GC-FID-2010 Conditions
   Injector Temperature: 230° C.
   Initial Column Temperature: 180° C.
   Initial Time: 1.75 minutes
   Temperature Ramp (1): 5° C./min to 200° C. Hold 1.75 minutes
   Temperature Ramp (2): 10° C./min to 240° C. Hold 7 minutes
   GC Column: Sigma-Aldrich SP-2560 100 m×0.25 mm ID×0.20 μm
   Detector Temperature: 240° C.
   Carrier gas: Hydrogen, LV=42.0 cm/sec
   Makeup Gas: Nitrogen, 30 ml/min
   FID Fuel gas: Hydrogen, 40 ml/min
   FID Oxidant gas: Compressed Air, 400 ml/min
viii) Electronic chromatograms are generated and saved for subsequent processing.

### 4) Data Processing of GC Chromatograms: Calculation of Raw Area Counts

i) The electronic chromatogram from the GC contains numerous peaks generated by the FID. The unknown peaks are compared to the known GLC FAME standard peaks and are processed or “picked” by a technician using the GCsolution post run software. Peak picking conventions can be determined by those of skill in the art, based on the teachings herein. GCsolution post run calculates the area under the curve, or area counts, for each peak. After the chromatogram is completely processed, a raw calculation of area counts and thus area percents (individual FA area counts/total FA area counts) is automatically done by Shimadzu's GCsolution post run software.

### 5) Calculation of Response Factors, Adjusted Area Counts, and Adjusted Area Percents

i) The GLC FAME standard was designed by the inventors and contains the weight percents of FAMEs as listed above which is roughly the FA composition found in human RBCs. The purpose of the GLC FAME standard is to serve as an external standard for our GC analysis and it allows for correction of variations in FID response factors to be made on a daily basis.
ii) The GLC FAME standard is included in each batch of unknowns run. In a perfect world, the GC would report out exactly the FA composition of the GLC FAME standard mixture. But this rarely happens. Instead, there are always slight differences between the known FA percent composition and the output of the analysis. This imprecision reflects day to day variation in “FID response”, that is, the number of “area counts” the electrometer is generating per unit mass of FAME burned in the FID. This variation in test result contributes to the day-to-day variability of the test, and controlling it reduces sample to sample variability.
iii) To account for daily response factor variation and to then adjust our unknowns for that variation, we put the raw area counts from the 22 peaks in GLC FAME standard output into an Excel macro. These data are copied from the GCsolution post run software and pasted into the macro. Area counts from the unknown samples included in that run are also copied and pasted into this file. This “pre-macro” is the file holding the raw data from each run.
iv) The output from the pre-macro is copied into a “conversion macro”. The area counts for a single FA (such as C16:0 (palmitic acid)) from the GLC FAME standard are assumed to be ‘true’ and are set to a response factor of 1.0. This means that no matter what the GC may say the weight percent of C16:0 is, the area counts are set to its known value of 15%. This establishes a value for the ratio of area counts to % composition which is then applied to the area counts for all of the other FAME peaks in the chromatogram. Thus, each FAME's area counts are divided by the ratio. This calculation creates new area counts for the unknown peak. The measured percent is divided by the target weight percent to generate the response factor. After the response factors are generated for each FA, they are applied to the samples by multiplying the response factor by the area counts of the unknown sample to yield the adjusted area counts, which are then converted into adjusted area percents for each FA. The FA adjusted percents can then be copied and pasted from the Conversion Macro to the study database.

### Fatty Acids Composition Used for Diagnostic Testing of Adolescent Depression

We compared the FA content of red blood cell membranes in adolescents admitted to the hospital for depression to those undergoing routine school/sports physical exams or who were being seen for minor illnesses or injuries. The study was a case-control design with blinded assessment of fatty acid composition. The data set included 283 subjects; 179 Caucasian and 104 African American. There were two subjects missing BMI, which reduced the data set to 281 adolescents (145 cases and 136 controls) for the purposes of generalized linear modeling. All fatty acid distributions were tested for normality, and appropriate transformations implemented as necessary. Next the fatty acids and BMI were standardized to have a mean (SD) of 0 (1). A multivariable logistic regression model using a combination of stepwise regression, Akaike's Information Criterion (AIC), and best subset selection was developed. The stepwise regression is used to determine the number of variables which minimizes the AIC. Then the best subsets of models with one less and one more variable than determined from stepwise are ranked by AIC and the minimum value is chosen as the best fitting model. The four demographic variables (BMI, Age, Sex, and Race) were forced into every iterative step. The hierarchy of stepwise selection was set to enter or remove a single fatty acid per iteration, but while allowing that fatty acid to have an interaction with race. P-values <0.05 were considered statistical significant. Analyses were performed using SAS version 9.2 (SAS Institute Inc., Cary, N.C.).

TABLE 2
Baseline Characteristics of Cases and Controls (N = 283)

| | | | |
|---|---|---|---|
| | Cases | Controls | |
| Variable | (n = 146) | (n = 137) | P-value* |

Demographics

| | | | | | | |
|---|---|---|---|---|---|---|
| Race: | Caucasian | 113 | (77)† | 66 | (48) | <0.0001 |
| | African American | 33 | (23) | 71 | (52) | |
| Sex: | Male | 64 | (44) | 69 | (50) | 0.27 |
| | Female | 82 | (56) | 68 | (50) | |

| | | | | | |
|---|---|---|---|---|---|
| Age [yr] | 15 | (14, 16)‡ | 15 | (14, 16) | 0.11 |
| Body mass index [kg/m²] | 23 | (20, 28) | 23 | (20, 26) | 0.93 |

Fatty Acids

| C14:0 - Myristic | | 0.31 | (0.26, 0.39) | 0.34 | (0.28, 0.40) | 0.12 |
|---|---|---|---|---|---|---|
| C16:0 - Palmitic | | 20 | (19, 21) | 20 | (20, 21) | 0.90 |
| C16:1t | - Palmitelaidic | 0.12 | (0.10, 0.14) | 0.10 | (0.09, 0.12) | <0.0001 |
| | - Caucasian | 0.12 | (0.11, 0.15) | 0.11 | (0.09, 0.12) | <0.0001 |
| | - African American | 0.10 | (0.09, 0.12) | 0.09 | (0.09, 0.12) | 0.21 |
| C16:1 - Palmitoleic | | 0.36 | (0.26, 0.50) | 0.27 | (0.21, 0.37) | <0.0001 |
| C18:0 - Stearic | | 16 | (14, 17) | 16 | (15, 17) | 0.35 |
| C18:1t - Elaidic | | 2.0 | (1.7, 2.2) | 1.9 | (1.7, 2.2) | 0.11 |
| C18:1 - Oleic | | 13 | (12, 14) | 13 | (12, 13) | 0.02 |
| C18:2n6t - Linolelaidic | | 0.32 | (0.29, 0.37) | 0.34 | (0.29, 0.39) | 0.17 |
| C18:2n6c - Linoleic | | 15 | (14, 16) | 15 | (14, 16) | 0.21 |
| C18:3n6 | - gamma Linolenic | 0.16 | (0.14, 0.18) | 0.16 | (0.14, 0.18) | 0.98 |
| | - Caucasian | 0.16 | (0.13, 0.18) | 0.17 | (0.15, 0.20) | 0.04 |
| | - African American | 0.15 | (0.14, 0.16) | 0.15 | (0.13, 0.16) | 0.44 |

| | | | | | |
|---|---|---|---|---|---|
| C20:1n9 - cis-11-Eicosaenoic | 0.15 | (0.12, 0.18) | 0.15 | (0.13, 0.18) | 0.49 |
| C18:3n3 - alpha Linolenic | 0.13 | (0.10, 0.15) | 0.09 | (0.07, 0.11) | <0.0001 |
| C20:2n6 - cis-11,14-Eicosadienoic | 0.27 | (0.25, 0.29) | 0.27 | (0.25, 0.30) | 0.32 |
| C20:3n6 - cis-8-11-14-Eicosatrienoic | 1.7 | (1.5, 1.9) | 1.6 | (1.4, 1.8) | 0.005 |
| C20:4n6 - Arachidonic | 19 | (18, 20) | 19 | (18, 20) | 0.69 |
| C24:0 - Lignoceric | 0.62 | (0.40, 0.75) | 0.62 | (0.43, 0.82) | 0.50 |
| C20:5n3 - Eicosapentaenoic (EPA) | 0.32 | (0.26, 0.41) | 0.33 | (0.26, 0.42) | 0.63 |
| C24:1n9 - Nervonic | 0.52 | (0.32, 0.67) | 0.50 | (0.35, 0.65) | 0.85 |
| C22:4 - Docosatetraenoic | 4.2 | (3.8, 4.5) | 4.1 | (3.8, 4.5) | 0.19 |
| C22:5n6 - n-6 Docosapentaenoic | 1.0 | (0.9, 1.1) | 1.0 | (0.9, 1.1) | 0.61 |
| C22:5n3 - n3 Docosapentaenoic | 2.1 | (1.9, 2.3) | 2.1 | (1.9, 2.3) | 0.07 |
| C22:6n3 - Docosahexaenoic (DHA) | 3.2 | (2.8, 3.7) | 3.4 | (2.8, 4.0) | 0.02 |

*Nonparametric Mann-Whitney U-test (Wilcoxon rank-sum) was used for continuous variables, and Chi-square test was used for categorical variables.
†n (%);
‡Median (IQR).

### Results

In univariate analysis shown in Table 2, seven FAs differed between cases and controls, including DHA which was lower in cases [3.2 (2.8,3.7)% vs 3.4 (2.8,4.0)%, p=0.02], and alpha-linolenic acid which was higher [0.13 (0.10,0.15)% vs 0.09 (0.07,0.11)%, p<0.0001; median (inter-quartile range)]. The data demonstrate that any one of palmitelaidic acid, palmitoleic acid, oleic acid, cis-8-11-14-eicosatrienoic, DHA, alpha-linolenic acid, and gamma-linolenic acid can be used alone to discriminate adolescents suffering from depression from those that are not suffering from depression. The multivariable logistic regression model adjusted for age, race, sex and BMI as demographic covariates and 10 FAs as metabolic covariates is shown in Table 3. The c-statistic is the concordance index which estimates the probability that the predictions and the outcomes are concordant. These results demonstrate that if the risk scores are calculated for two adolescents (one with depression and one without depression) there is an 88% probability that the depressed adolescent will have a higher risk score based on this invention (Table 4). Other useful measures for assessing a diagnostic test are sensitivity, specificity, and the positive likelihood ratio (which summarizes the former two measures into one number). These findings support the hypothesis that depression is associated with altered membrane FA composition, and suggest that RBC FA composition can serve as a diagnostic test for adolescent depression.

TABLE 3
Estimated parameter estimates in depressed adolescents per standard deviations

| | | | | SE | |
|---|---|---|---|---|---|
| Variable | Transformation | SD | Est. (β) | (β) | P-value |
| BMI | Inverse | 5.1 | 0.29 | 0.19 | 0.12 |
| Age (per 1 year) | | | −0.12 | 0.12 | 0.29 |
| Female | | | 0.06 | 0.35 | 0.85 |

Fatty Acids (% of total FA)

| *Intercept: | Caucasian | | | 2.45 | 1.81 | 0.18 |
|---|---|---|---|---|---|---|
| | African American | | | 1.32 | 1.85 | 0.48 |
| Linolelaidic acid | | Ln | 0.08 | −0.72 | 0.19 | 0.0002 |
| *Palmitelaidic acid: | Caucasian | Ln | 0.03 | 1.30 | 0.31 | <0.0001 |
| | African American | | | 0.39 | 0.25 | 0.12 |
| alpha-Linolenic acid | | Square root | 0.04 | 1.08 | 0.27 | <0.0001 |
| Docosatetraenoic acid | | Squared | 0.55 | 0.64 | 0.21 | 0.003 |
| Myristic acid | | Inverse | 0.10 | 1.13 | 0.23 | <0.0001 |
| *gamma-Linolenic acid: | Caucasian | Ln | 0.03 | −1.08 | 0.30 | 0.0003 |
| | African American | | | 0.07 | 0.29 | 0.80 |

| | | | | | |
|---|---|---|---|---|---|
| Linoleic acid | Inverse | 1.75 | −0.72 | 0.22 | 0.001 |
| Arachidonic acid | None | 1.57 | 0.49 | 0.21 | 0.02 |
| Palmitoleic acid | Ln | 0.17 | 0.75 | 0.27 | 0.005 |
| cis-8-11-14-Eicosatrienoic acid | Ln | 0.33 | 0.23 | 0.18 | 0.21 |

*Significant racial interaction effects;
Ln, Natural logarithm.

TABLE 4
Diagnostic Test Characteristics
All models adjusted for the following confounding factors: BMI, age, sex, race

| | | | | Area |
|---|---|---|---|---|
| | | | Positive | under |
| | | | Likelihood | ROC |
| | Sensitivity | Specificity | Ratio | curve |
| Saturated | | | | |
| Myristic, Stearic, Palmitic, Lignoceric | 69% | 58% | 1.6 | 73% |
| Monounsaturated | | | | |
| Palmitoleic, Oleic, cis-11-Eicosaenoic, Nervonic | 71% | 59% | 1.7 | 71% |
| Trans | | | | |
| Palmitelaidic*, Linolelaidic, Elaidic | 66% | 69% | 2.1 | 77% |
| Polyunsaturated n-6 + n-3 | 68% | 64% | 1.9 | 77% |
| Polyunsaturated n-6 | | | | |
| gamma-Linolenic*, Docosatetraenoic, cis-8-11- | 69% | 65% | 2.0 | 74% |
| 14-Eicosatrienoic, Linoleic, Arachidonic, n-6 | | | | |
| Docosapentaenoic, cis-11,14-Eicosadienaic | | | | |
| Polyunsaturated n-3 | | | | |
| alpha-Linolenic, Docosahexaenoic (DHA), n-3 | 69% | 67% | 2.1 | 74% |
| Docosapentaenoic, Eicosapentaenoic (EPA) | | | | |
| Most predictive fatty acid set | | | | |
| Myristic, Palmitoleic, Palmitelaidic*, Linolelaidic, | 75% | 75% | 3.0 | 88% |
| gamma-Linolenic*, Docosatetraenoic, cis-8-11- | | | | |
| 14-Eicosatrienoic, Linoleic, Arachidonic, alpha- | | | | |
| Linolenic | | | | |

*Racial interaction between blacks and whites, meaning each race has it's own beta coefficient

Using a combination of Myristic, Palmitoleic, Palmitelaidic, Linolelaidic, gamma-Linolenic, Docosatetraenoic, cis-8-11-14-Eicosatrienoic, Linoleic, Arachidonic, alpha-Linolenic fatty acids t and no adjustment for the confounding factors (BMI, age, sex, race) the following results are obtained:

Area under the ROC curve=86%

### Sensitivity=74%

### Specificity=72%

### Positive Likelihood Ratio=2.7

For perspective, the Framingham Risk Score is a tool used widely to predict risk for cardiovascular disease. It is based on 7 factors: age, sex, cholesterol, HDL-cholesterol, blood pressure, smoking status, and diabetes. Typically, the Framingham Risk Score has a sensitivity of 0.75, a specificity of 0.73, a positive likelihood ratio of 2.9 and an area under the ROC curve (AUC) of 0.81. Thus, the 10-fatty acid combination (Myristic, Palmitoleic, Palmitelaidic*, Linolelaidic, gamma-Linolenic*, Docosatetraenoic, cis-8-11-14-Eicosatrienoic, Linoleic, Arachidonic, and alpha-Linolenic), with its AUC of 0.88 (considering confounding factors) or 0.86 (without considering confounding factors), is substantially better at discriminating adolescents with and without depression than the Framingham score is at distinguishing adults who've had a heart attack and from those who have not.

We claim:

1. A method for assessing adolescent depression in a subject, comprising
(a) measuring an amount of one or more fatty acids in a tissue sample from a subject at risk of suffering from adolescent depression;
(b) comparing the amount of the one or more fatty acids to a control; and
(c) determining a probability that the adolescent subject has depression based on the comparison.

2. The method of claim 1, wherein the one or more fatty acids are selected from the group consisting of myristic acid, palmitic acid, palmitelaidic acid, palmitoleic acid, stearic acid, elaidic acid, oleic acid, linolelaidic acid, linoleic acid, gamma linolenic acid, cis-11-eicosenoic acid, alpha-linolenic acid, cis-11,14-eicosadienoic acid, cis-8-11-14-eicosatrienoic acid, arachidonic acid, lignoceric acid, eicosapentaenoic acid (EPA), nervonic acid, docosatetraenoic acid, n-6 docosapentaenoic acid, n-3 docosapentaenoic acid, and docosahexaenoic acid (DHA).

3. The method of claim 1, wherein the one or more fatty acids are selected from the group consisting of linolelaidic acid, palmitelaidic acid, alpha-linolenic acid, myristic acid, gamma-linolenic acid, docosatetraenoic acid, palmitoleic acid, linoleic acid, arachidonic acid, and cis-8-11-14-eicosatrienoic acid.

4. The method of claim 3, wherein the method comprises measuring the amount of two or more of the fatty acids.

5. The method of claim 4, wherein the method comprises measuring the amount of three or more fatty acids.

6. The method of claim 1 wherein the tissue sample comprises red blood cells, whole blood, serum, platelets, white blood cells, plasma or plasma phospholipids.

7. The method of claim 1 wherein measuring an amount of one or more fatty acids comprises measuring a percent total of the individual one or more fatty acids as a percent of total fatty acids from the tissue.

8. The method of claim 7, wherein comparing the amount of the one or more fatty acids to a control comprises
(a) multiplying the percent total of the individual one or more fatty acids by a predetermined risk factor coefficient to produce an individual fatty acid score; and
(b) summing the individual fatty acid scores to produce a risk score.

9. The method of claim 7, further comprising subjecting the percent total of the individual one or more fatty acids to an analysis selected from the group consisting of generalized models, multivariate analysis, and time-to-event survival analysis, to produce a modified risk score; wherein the modified risk score is used to determine the probability for adolescent depression.

10. A method for determining the fatty acid composition of a tissue membrane, comprising
(a) methylating fatty acids in the tissue sample to produce fatty acid methyl esters (FAMEs) by treatment with a boron-trifluoride methanol solution;
(b) extracting FAMEs from the tissue sample using a hexane solvent;
(c) separating the FAMEs by gas chromatography in a fused silica capillary column;
(d) determining retention times of the FAMEs in the column by means of a flame ionization detector (FID); and
(e) comparing the retention time to a FAME standard; and
(f) calculating FAME response factors for each FAME in the sample based on a FAME standard.

11. The method of claim 10, wherein calculating response factors for each FAME comprises:
(i) identifying an area count for a reference fatty acid with a known percent composition in the FAME standard and calculating a ratio of counts to percent;
(ii) applying the ratio to correct area counts for the fatty acids in the FAME standard; and
(iii) calculating a response factor for each of the fatty acids.
